(19) United States
(12) Patent Application Publication
(10) Pub. No.: US 2015/0305879 A1
(43) Pub. Date: Oct. 29, 2015

(54) PROSTHESIS INTERSOMATIC OF LUMBAR AND SACRAL FUSION APPLIED IN THE PATHOLOGY TREATMENT OF SAGITTAL MISALIGNMENT TYPE

(71) Applicant: MDT INDÚSTRIA COMÉRCIO IMPORTAÇÄO E EXPORTAÇÄO DE IMPLANTS S.A., Rio Claro (BR)

(72) Inventor: Luiz Henrique Mattos PIMENTA, Sao Paulo (BR)

(73) Assignee: MDT INDÚSTRIA COMÉRCIO IMPORTAÇÄO E EXPORTAÇÄO DE IMPLANTES S.A., Rio Claro (BR)

(21) Appl. No.: 14/260,963

(22) Filed: Apr. 24, 2014

(51) Int. Cl. A61F 2/44

(52) U.S. Cl. CPC A61F 2/442

(57) ABSTRACT

IMPROVEMENT IN PROSTHESIS INTERSOMATIC OF LUMBAR AND SACRAL FUSION APPLIED IN THE TREATMENT OF PATHOLOGY OF TYPE MISALIGNMENT SAGITTAL represented by a solution in evolutionary medicine, especially in the segment of medicine known as orthopedics, and more specifically in the area relevant to the treatment of diseases in the region of the lumbar spine and sacrum, where with the introduction of the new prosthesis (Pr) is made possible as an alternative treatment to the sagittal alignment, being preferably an alternative treatment for osteotomy technique of pedicle subtraction (OSP), given that minimizes the occurrence Complications of neurological nature, being that the monolithic body (1) of the prosthesis (Pr) is to present their top and bottom surfaces (4) provided with sharp angulation orthogonal.

### FIELD OF APPLICATION

The present invention patent in title as above and object of description and claim this credit title is a novelty solution in medicine field, particularly in the area of medicine known as orthopedics, and more specifically in the relevant area to the treatment of diseases in the region of the lumbar spine and sacrum.

However the improvement in prosthesis intersomatic meets all the conditions of a normal cage, being indicated in treatment of pathology as intersomatic arthrodesis with imbalance in lordosis.

### DEMAND OF THE NOVELTY

In this patent, a complement of relevant notable importance to the matters disclosed and claimed in the dispute of invention patent PI-1002012-8, by title “CAGE SYSTEM FOR IN-TERVERTEBRAL ANTERIOR FUSION OF THE SPINAL COLUMN” deposited on Feb. 10, 2010.

Whereas the novelty cited, particularly the term “cage” can be understood as a “prosthesis intersomatic of the lumbar and sacral fusion” this improvement was applied to structural body of this prosthesis gives improvements from a functional standpoint, especially when you have a view of your application to the pathology of the lumbar spine and sacral what is technically known as “misalignment sagittal”.

The inventor points out that the introduction of improved “prosthesis intersemiotic for lumbar and sacral fusion” originally revealed in the patent of invention PI-1002012-8, although I will leave more broad spectrum of application of this type of prosthesis, this evolutionary solution does not give up of all benefits previously listed in the text of the descriptive report of patent originally filed, whose valuation will be revealed in the development of the body of this descriptive report.

### PARADIGM OF THE NOVELTY

In accordance with the as revealed in the topic of demand of the novelty the applicant has elected as development paradigm the pathology of the lumbar spine and sacral technically known as “misalignment sagittal”.

### REQUIREMENTS OF THE NOVELTY

In accordance with the demand and paradigm of the novelty the applicant conceived of an nonobviousness “improvement in prosthesis intersomatic for lumbar and sacral fusion applied in the treatment of pathology of type misalignment sagittal” provided with novelty associated of inventive step, because it is not so obvious or clear of other known solutions in the state of the technique for the treatment of said pathology.

In addition the “novelty” is equipped with utility, being economically viable and, therefore, given the rigor of the patenteability requirements, notably as patent of novelty, as provisions in the dictates of articles 8 and 13 of Law 9,279.

### FUNDAMENTALS OF TECHNICAL

In order to provide accuracy, and consolidate the context depicted on the topics in introductory framework will be presented with a discussion of the state of the art dictation solutions developed and widely used for the treatment of the disease known as “misalignment sagittal”, where after critical analysis of these, once exposed to professionals with expertise in medicine, can identify their limiting aspects, thus consolidating the identification of needs of demands for improvement was applied in “prosthesis intersomatic of lumbar and sacral fusion”

A. Understanding the pathology of misalignment sagittal: The accented misalignments of the spinal column bring as negative aspect to the individual's health the fact that if you lose the body efficiency energy for which there are postural compensation in order to remain in an upright position, balanced and without pain, since the posture of the pelvis, hip, and lower limbs of the human body is usually modify in an attempt to minimize the deformity of the spine and the bad sagittal alignment, as can be seen in FIG. 1, where they are presented with four different situations of body posture to know:
Position of the erect body with ideal balance (P1);
Position of the erect body with compensating balance (P2);
Position of the arched body with compensating balance (P3); and
Position of the arched body with previous compensating (P4).

B. The treatment of the sagittal alignment: the treatment of this type of pathology is done commonly by means of specific techniques, based on Osteotomy of Pedicle Subtraction—PSO, for example, that it is a surgical treatment with the main objective to promote the decompression of the spinal canal and the restoration of the balance sagittal and coronal planes of the vertebral column.

In its turn, the success of this intervention is directly connected to aspects such as:
Type of lesion;
   Lesion time and the surgical treatment.
The patient's age; and
Clinical conditions of the patient.

C. Critical Analysis of treatment by osteotomy of pedicle subtraction—OPS: although the surgical results of OPS is favorable, reports indicate that there are any complications of neurological nature, being that the rate of these occurrences is considered among physicians very high.

### THE PROPOSAL OF THE NOVELTY

The Objective: aware of the gap that exists for the current treatment of the sagittal alignment, the inventor was the development of an inventive solution in the form of a “prosthesis intersomatic for lumbar and sacral fusion”, which allows the solver the problem pointed to occurrence of neurological complications after surgery.

B. Distinguishing Feature: to make feasible the beneficial described the inventor has adopted as a paradigm of development the technological base disclosed and claimed in the patent of invention PI-1002012-8, title “CAGE SYSTEM FOR INTERVERTEBRAL FUSION ANTERIOR OF SPINE” deposited on Feb. 10, 2010, where tight synthesis his body, in the form of monolithic material presents specific openings for the introduction of mounting bolts, being that its upper and lower surface is designed to be accommodated in the areas below and above the superior and inferior vertebrae respectively. On this way once introduced the prosthesis, occurs a gap between the vertebrae until then compressed.

The solution is distinctive by the fact that their structure body, becomes the present section orthogonal cutting with angulation (upper and lower), with values accentuated, in the range between 30 and 75°.

### DESCRIPTION OF THE FIGURES

The complement to this description in order to get a better understanding of the characteristics of this invention and in accordance with a preferred form of practical implementation of the same, accompanies the description in Annex, a set of drawings, where, so exemplified, but not limited to, are represented:

FIG. 1 is a schematic representation of the stages Impairment of the column of an individual, showing since the position of erect body with body balance ideal until the position of body arched with previous imbalance;

FIG. 2 is a representation in perspective view of prosthesis intersemiotic for lumbar and sacral fusion improved.

FIG. 3 is a representation in perspective view “A” of prosthesis intersemiotic for lumbar and sacral

Fusion improved.

FIG. 4 is a representation in perspective view “B” of prosthesis intersemiotic for lumbar and sacral fusion improved;

FIG. 5 is a representation in perspective view “C” of prosthesis intersemiotic for lumbar and sacral fusion improved;

FIG. 6 is a representation in cut “XX” of prosthesis intersemiotic for lumbar and sacral fusion improved.

FIG. 7 is a representation in cut “YY” of prosthesis intersemiotic for lumbar and sacral fusion improved.

FIG. 8 is a representation in a side view of screw fixation of prosthetic intersomatic lumbar and sacral fusion improved at the vertebra near the bottom;

FIG. 9 is a representation in a frontal view of screw fixation of prosthetic intersomatic lumbar and sacral fusion improved at the vertebra near the bottom;

FIG. 10 is a representation in a cut view “ZZ” of screw fixation of prosthetic intersomatic lumbar and sacral fusion improved at the vertebra near the bottom; and

FIG. 11 is a representation in a frontal view of screw fixation of prosthetic intersomatic lumbar and sacral fusion improved at the vertebra near the bottom;

### DETAILED DESCRIPTION

The following detailed description should be read and interpreted with reference to drawings, representing a form of preferred implementation for improvement in “prosthesis intersomatic of lumbar and sacral fusion improved”, and is not meant to limit the scope of the invention, this rather limited only to depicted in table claiming.

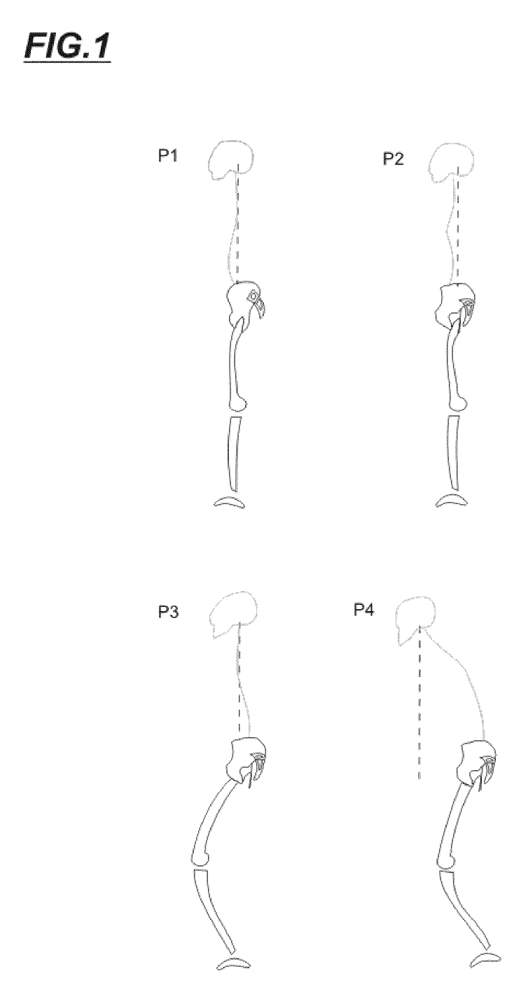

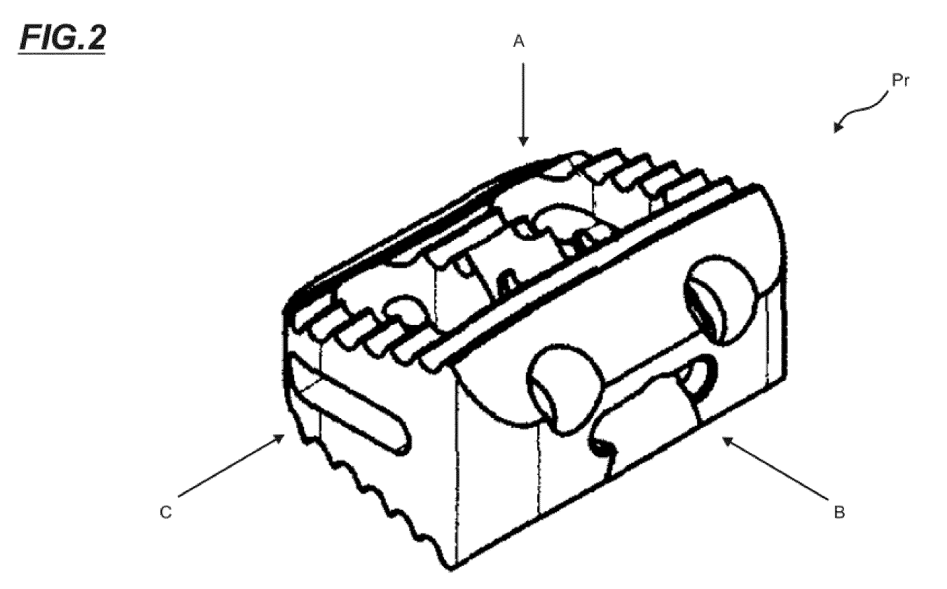

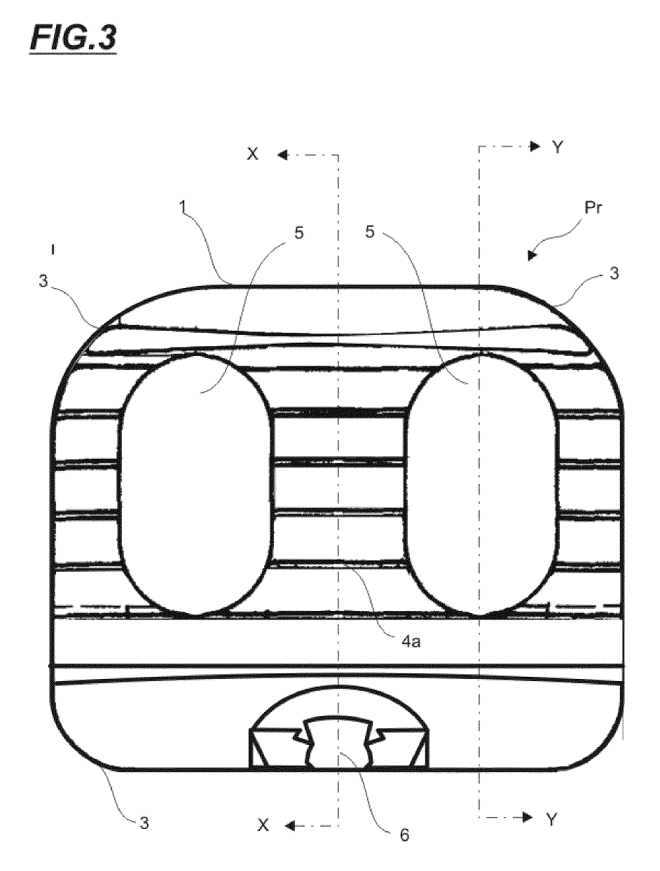

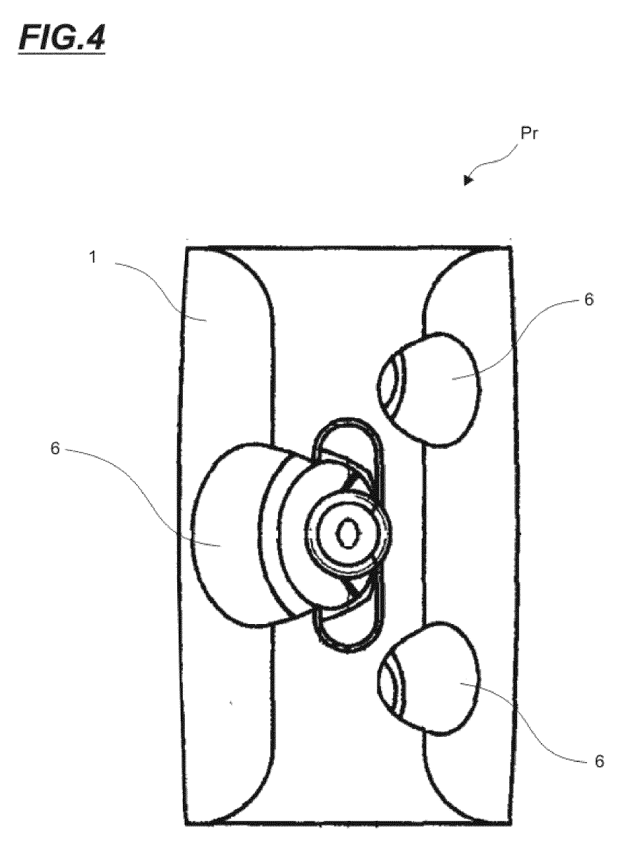

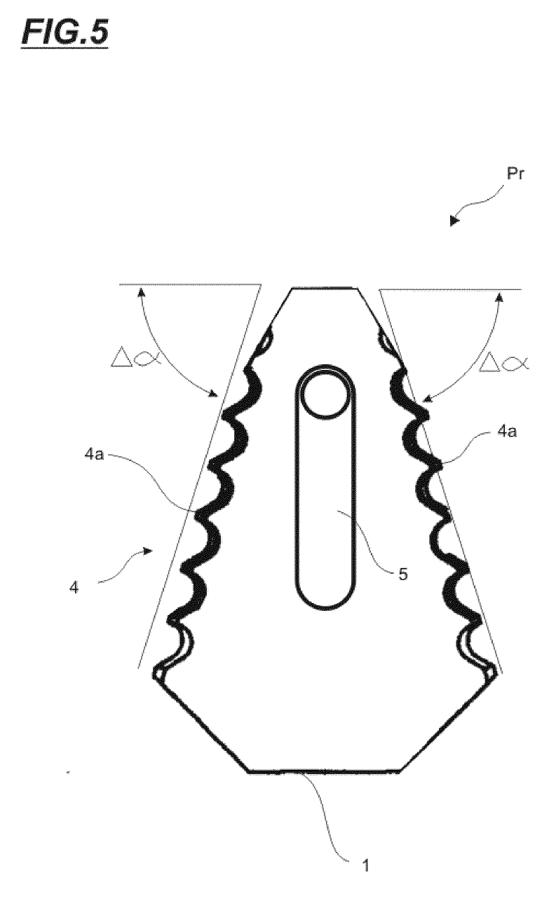

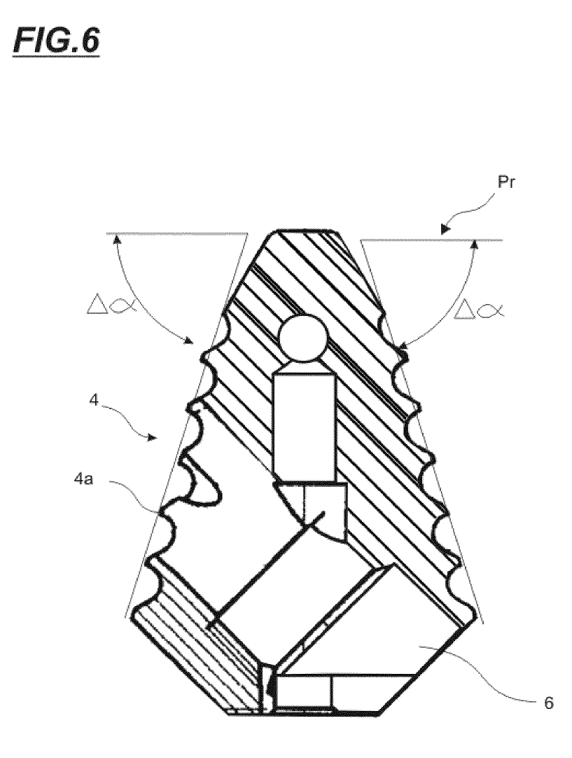

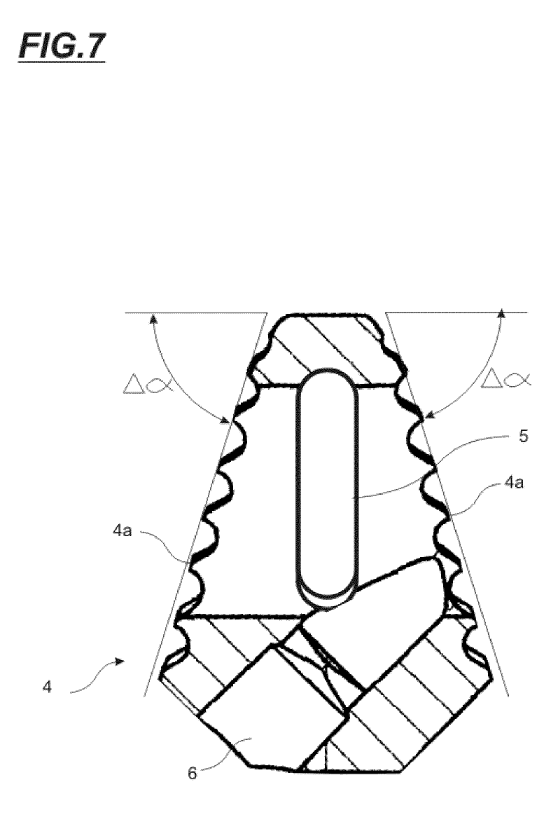

a. The technology base: as evidenced in FIGS. 1 to 7, the prosthesis (Pr) is formed by a monolithic body (1), of appropriate size and shaped like a vertebra of the lumbar spine and sacrum, being that this can be made of metallic material, preferably titanium and alternatively in material PEEK->Poly-ether-ether-ketone.

Your constructive concept is translated by this body (1) submit rounded corners (3), being defined through openings (5). In addition are defined directional holes with angulation (6), to which are applied the mounting bolts (Pa). In turn the monolithic body (1) has upper and lower surfaces (4), which are defined grooves (4a).

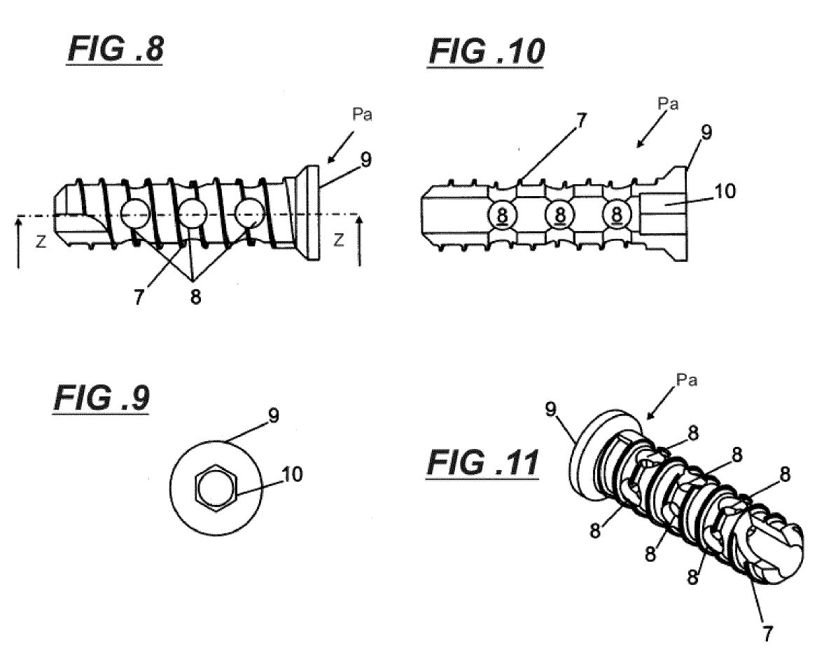

In ultimate bolts (Pa), which in a way in which preferential are presented in FIGS. 8 to 11, can be described as bolts of primary fixation, which have threaded body (7), provided with at least three holes perpendicular (8), where at the end of this body (7) is defined a head (9), preferably round, which presents a hex recess (10).

Distinguishing Feature: as evidenced in FIGS. 1 to 7, the monolithic body (1) presents their surfaces (4), upper and lower, provided with an angular variacão angle variation (Act) with accented values, in the range between 30 and 75°.

Technical Effect obtained: when putting the prosthesis surgically(Pr) in the region of intervention in the spine, more Specifically in area where occurs the pathology of “misalignment sagittal” the vertebra with problem is shifted up and when loose passes to compress this same prosthesis (Pr), in relation to the lower vertebra, due to the weight of the upper vertebra.

Once properly positioned the prosthesis (Pr), is applied to the bolt assembly (Pa) thereby promoting the stability of the system formed by lower vertebra+prosthesis (Pr)+upper vertebra, being fixed a bolt near the lower vertebra with centralization in the middle of the body (1) and two screws near the upper vertebra, all through holes signals with angulation (6).

The choice of a preferred implementation of the invention object of claim in this credit title, described in this topic in detail of the novelty is provided as an example only. Amendments, modifications and variations can be made for other forms of “prosthesisintersomatic of lumbar and sacral fusion improved” by those with skill in the art without, however deviate from the objective revealed in elections of this patent, which is exclusively defined by the claims attached hereto.

Within this context it is important to explain that due to the special features of each patient, there is the possibility of countless you specify dimensional for this type of prosthesis (Pr) improved, thus characterizing a product whose main dimensions are parameterized.

Faced with this scenario it is possible to say that all and any size of parameters, notably the parameter for the angulation orthogonal set for the superfies upper and lower (4) monolithic body (1) fits within the same concept nonobviousness led in claiming.

Even within the context of paragraph (23), it is also possible to claim that the material of manufacture of prosthetic devices (Pr) in addition to the traditional titanium, may be performed by any types of metallic materials, polymer and in contemporary composite materials (metal+polymers, among others).

It is by that was described and illustrated that the “IMPROVEMENT IN PROSTHESIS INTERSOMATIC OF LUMBAR AND SACRAL FUSION APPLIED IN THE TREATMENT OF PATHOLOGY OF TYPE MISALIGNMENT SAGITTAL” now claimed fits the rules governing patent of novelty in the light of the Law of Industrial Property, deserving from what has been stated above and as a result, their privilege.

1. AN IMPROVEMENT IN PROSTHESIS INTERSOMATIC OF LUMBAR AND SACRAL FUSION APPLIED IN THE TREATMENT OF PATHOLOGY OF TYPE MISALIGNMENT SAGITTAL, where a prosthesis (Pr) is formed by a monolithic body (1), provided with rounded corners (3), with superfies upper and lower (4) provided with grooves (4a) and also with openings passers-by (5) and holes signals with angulation (6) to which are applied the mounting bolts (Pa), being that the improvement is characterized by areas (4), upper and lower, submit each one angle variation (Δα) in the range between 30 and 75°.
